(19) United States
(12) Patent Application Publication
(10) Pub. No.: US 2024/0209306 A1
(43) Pub. Date: Jun. 27, 2024

(54) Methods and Systems for Optimizing Culture Conditions in a Culture Process

(71) Applicant: POW Genetic Solutions, Inc., Emeryville, CA (US)

(72) Inventors: Shannon HALL, San Francisco, CA (US); Ouwei WANG, San Francisco, CA (US); Brian LEE, San Francisco, CA (US); Charles EMRICH, San Francisco, CA (US)

(21) Appl. No.: 18/287,287

(22) PCT Filed: Dec. 21, 2022
(86) PCT No.: PCT/US22/53733

§ 371 Date: Oct. 17, 2023

Related U.S. Application Data
(60) Provisional application No. 63/292,397, filed on Dec. 21, 2021.

(51) Int. Cl. C12M 1/36; C12M 1/00; C12M 1/34; C12N 1/20

(52) U.S. Cl. CPC C12M 41/48; C12M 27/20; C12M 29/06; C12M 41/12; C12M 41/26; C12M 41/32; C12M 41/34; C12N 1/20

(57) ABSTRACT

Methods and systems for iteratively improving culture condition for at least one culture output in a culture process. The methods comprise: a) providing a continuous culture of cells in a bioreactor, wherein the cells are cultured under culture conditions that comprise a set of culture parameters. and wherein the culture of cells produces at least one culture output at a first measure: b) while maintaining the cells in continuous culture, iteratively testing different culture conditions by: (i) selecting a new culture condition comprising a new set of culture parameters predicted to improve the measure of the culture output: (ii) perturbing the cells with the new culture conditions: and (iii) determining a new measure of the culture output: wherein testing is iterated to produce a plurality of improvements in the measure of culture output. The iterative process can be performed by an automated artificial intelligence.

### REFERENCE TO RELATED APPLICATIONS

This application claims the benefit of the filing date of U.S. provisional application 63/292,397, filed Dec. 21, 2021, the contents of which are incorporated in their entirely by reference.

### STATEMENT AS TO FEDERALLY SPONSORED RESEARCH

None

### THE NAMES OF THE PARTIES TO A JOINT RESEARCH AGREEMENT

None

### SEQUENCE LISTING

None

### BACKGROUND

The booming biomanufacturing and synthetic biology industries rely on bioreactors (also called fermenters) to bio-transform raw materials into value-added products. Biomanufacturing is a nascent sector that can be leveraged to produce a large share of the global economy's physical materials, not only with improved performance but also with better environmental footprint to combat climate change. Recent estimates place the total annual economic impact of synthetic biology at $2-4 Trillion by 2040. Two thirds of this impact are outside of healthcare—in agriculture, consumer products, biomaterials, biochemicals, bioenergy, and many other areas. Among all potential applications of synthetic biology, the biomanufacturing of alternative proteins, biomaterials, biochemicals, and biofuels will account for the largest growth the next 10 to 20 years.

While biomanufacturing may be one of the most technologically complex industries, the underlying production process, batch fermentation, has not changed in decades. Batch reactors are slow, repetitive, and serial in nature. Bioreactors are extremely expensive to acquire, in addition to having long lead times. Owing to the prohibitive price and limited availability of bioreactors, “tank-time” has become the biggest bottleneck for fermentation process (bioprocess) optimization. For example, using the Design of Experiment (DoE) approach to identify the most optimal process conditions could requires hundreds of bioreactor runs, each with a different set of operating conditions. Assuming that each run, on average, takes a week, one hundred runs with a single bioreactor will take two full years to realize. Even established companies with fleets of bioreactors are limited by the “tank-time” they can spend per project, and can be forced to reduce the number of conditions to be tested. Many projects are “shelved” due to failure to show improvement over time during bioprocess scale up. Similar limitations also exist in bioreactor strain screening, where, owing to the limited tank-time, only one “baseline” process condition is used to screen all strains, which often causes the true optimal strain to perform terribly due to a sub-optimal bioprocess conditions. The limitation in tank-time due to high-bioreactor cost results in inadequate process optimization and rampant false negative outcome in strain screening, which consequently lead to missed financial opportunities and suboptimal performance at large scale.

### SUMMARY

Disclosed herein is a methodology for generating-high throughput fermentation process data for rapid process optimization and strain screening against multiple process parameters. This high throughput fermentation platform leverages an autonomous agent such as machine learning (ML) empowered real-time optimization based on specific resource consumption rates (mass of resource consumed per hour per cell), specific production rates (mass of product produced per hour per cell), and production economics. This real-time automatic optimization uses a robust continuous fermentation platform. Replacing the traditional fed-batch bioreactor systems with a continuous bioreactor minimizes equipment downtime, increases volumetric productivity, and reduces operational cost. Unlike batch fermentation, which only tests a single condition per tank run, the continuous bioreactors enable temporal multiplexing of process condition testing. One can test as many conditions as needed throughout the run. This results in a five- to twenty-fold reduction in time needed for process optimization, and in number of tanks required to complete an optimization campaign. The disclosed technology provides a massive productivity boost as measured by data generated per tank per day, addressing a major pain point in the biomanufacturing industry, and breaking the optimization bottleneck of synthetic biology. Successful implementation of continuous optimization allows high-throughput automation technology and future developments in continuous downstream processing.

A first step in this high-throughput method is to determine the baseline strain stability in a continuous bioreactor (e.g., turbidostat) and the baseline productivity in a (fed)batch reactor. The continuous run tests the strain stability and provides the number of generations that can be run in a continuous system before productivity decay, which defines the range of time that a continuous reactor can operate. The (fed)batch reactor run determines the initial growth rate, productivity, and yield prior to optimization. A strain with constitutive or inducible expression of the production pathway is cultured in a turbidostat with constant biomass concentration and fermentation input parameters. For inducible processes, inducer/repressor molecules are added/eliminated after initial growth to induce the production pathway. After 24 hours the turbidostat system reaches a steady-state where constant product titer and specific productivity are observed. Strain breakage will cause a gradual titer and productivity decay over time, which will be observed and measured via offline sample analysis. Strain stability will be accessed based on the time elapsed until strain breakage. As an unstable strain is unsuitable for commercial production, this step produces a quick go/no-go and can save time and money before further unfruitful development is implemented.

A second step is to optimize for the best culturing conditions for the production organism, as a robust growth environment serves as the foundation for production. A turbidostat can be used to optimize the growth rates. A strain with constitutive or inducible expression of the production pathway can be cultured in a turbidostat with constant biomass concentration but varying fermentation input parameters. After 24 hours the turbidostat system reaches a steady- state where all nutrients are in excess and the production organism reaches its maximum specific growth rate. Different input fermentation parameters will be applied to perturb the system from the current steady state to move to a new steady-state. The new steady-state will have a new maximum specific growth rate that is different than the previous specific growth rate. This process will continue until a maximum specific growth rate is identified, or the generation time define by step (1) is reached. Input fermentation parameters are implemented to perturb the system. The inputs can be changed in step-wise or a sinusoidal manner, instead of reaching a steady state. The rate of change or the trend of change in specific growth rate between two sets of input fermentation parameters can be compared to identify the ranking of the two conditions. The process conditions tested can be randomized or repeated to increase the statistical power of the results. This step could be optional for certain common production hosts such as Escherichia coli, Bacillus spps, Pichia pastoris, Saccharomyces cerevisiae, as the growth range for most common production platforms are already known.

A third step is to optimize for the best production condition for the production organism. This approach not only scans for an array of fermentation conditions for the highest productivity and yield but also represent a more comprehensive method for strain screening as it is no longer restricted by a single screening condition such as in the fed-batch mode. A chemostat can be used to optimize for specific productivity and yield. For inducible strains, inducer/repressor molecules are added/eliminated after initial growth to induce the production pathway. After 24 hours the chemostat system reaches a steady-state where all nutrients except one are in excess and the production organism reaches a steady state. In a steady-state, the rate of addition of this rate-limiting nutrient will define the growth rate of the system. Different input fermentation parameters will be applied to perturb the system from the current steady-state (State 1) to move to a new steady (State 2-N) state. The new steady-state will have a new maximum specific productivity and yield that is different than the previous specific productivity and yield. This process will continue until the maximum specific productivity and yield are identified, or the generation time to strain stability is reached. When input fermentation parameters are implemented to perturb the system, the inputs can be changed in step-wise or in a sinusoidal manner. The process conditions tested can be randomized or repeated to increase the statistical power of the results. The rate of change or the trend of change in specific growth rate between two sets of input fermentation parameters can be compared to identify the ranking of the two conditions.

The high-throughout fermentation data generation can be automated by a computational algorithm such as a machine learning based algorithm. For example, reinforcement learning, which is one of three basic machine learning paradigms, can be used to take actions during the continuous turbidostat or chemostat process in order to maximize the desired fermentation output parameters. Other mathematical optimization methods could be also applied such as the dynamic programming method, gradient descent method, and the simplex algorithm. This machine learning aided approach would drastically reduce the conditions need to be tested and also identify optimal conditions that may be contrary to human intuition.

A fourth step is to validate the optimized parameters in fed-batch bioreactors. The newly optimized fermentation input parameters such as pH, temperature, dissolved oxygen, carbon feed rate, nitrogen feed rate, other chemical composition of the medium will be tested to verify and validate the optimization pipeline resulted in a significant improvement of productivity and/or production economics.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an exemplary overall process for optimizing culture conditions for cell growth and production of a product by cells cultured in a bioreactor.

FIG. 2 shows three process stages in more detail.

FIG. 3 shows a more detailed, exemplary version of a process for optimization of culture conditions.

FIG. 4 shows a schematic of reinforcement learning.

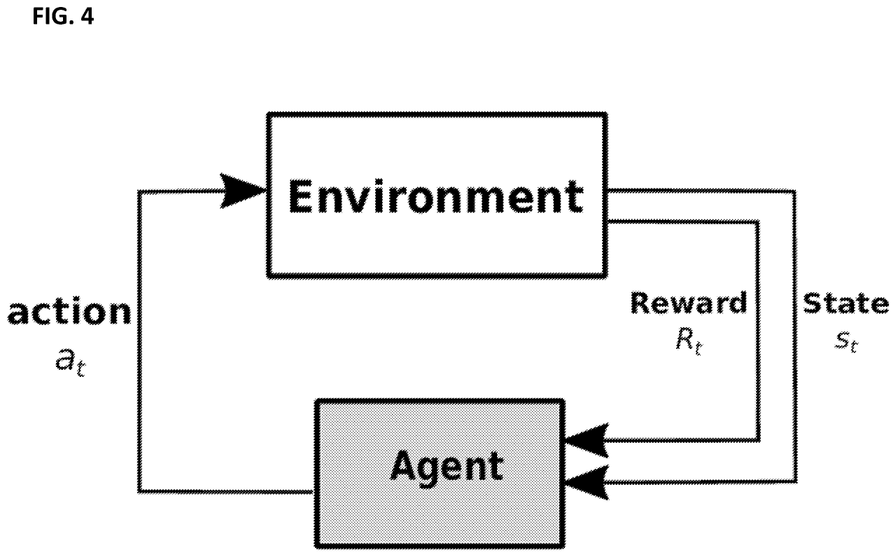

FIG. 5 shows results from a turbidostat in which temperature and PH are maintained constant over time and cells are maintained constant concentration (AU).

FIG. 6 shows a comparison between an optimization process performed in continuous culture and a process performed in batch culture. In continuous culture, a plurality of different culture conditions can be tested over the course of a single run whereas, under batch conditions each run is characterized by single culture condition.

FIG. 7 shows progress of an optimization experiment in which an intelligent agent selects new culture conditions predicted to improve culture output, in which the prediction is based on prior results.

FIGS. 8A and 8B show an exemplary bioreactor.

FIG. 9 shows an exemplary computer system.

### DETAILED DESCRIPTION

### I. Introduction

Provided herein are methods of optimizing culture conditions for a desired culture output. In contrast to prior methods, which relied on (fed)-batch culture methods, methods of optimization described herein are performed in continuous culture.

### A. Continuous Culture

A primary difference between batch culture and continuous culture is that batch culture is a closed system in which cell culture, such as fermentation, is carried out with a fixed amount of nutrients, while continuous culture is an open system in which nutrients are continuously added to the culture. Batch culture is characterized by initial rapid growth of cells followed by a plateau after nutrients are used up and cell density inhibits further growth. Continuous culture is characterized by a steady growth rate and/or a steady rate of culture output, as nutrients are continuously added to the system and cell density is maintained by removal of excess cells from the culture.

Culture processes embrace any culturing of cells for producing a culture output. These processes include, without limitation, processes referred to as “fermentation” and “cell culture.” “Fermentation” generally refers to enzymatic conversion of molecules into different molecular species (e.g., conversion of sugar into ethanol), typically by single-celled organisms. “Cell culture” generally refers to the culture of single cells, e.g., mammalian cell or insect cells, for the production of a product, such as a polypeptide or a value-added organic compound. The processes further embrace synthetic biology methods, in which cells are engineered to produce molecular outputs, for example, by expression within the cell of enzymes along a synthetic pathway.

### B. Bioreactor

Culture of cells is typically performed in a bioreactor. Bioreactors are containers or vessels configured to contain culture medium and which comprise elements to maintain or alter various culture parameters. This includes, for example, thermal regulators and thermometers to measure temperature, sources of nutrients and sensors to measure culture parameters, such as pH meters. Bioreactors also can contain stirring devices, such as paddles and ports for input of nutrients and other chemicals and for output of culture medium, which may include, cells. Bioreactors can contain feeds for the addition of nutrients and other molecules as well as an influent to remove culture liquid from the bioreactor.

Two types of bioreactors used for continuous culture include the turbidostat and the chemostat. In both cases nutrients and other chemicals are added to the cell culture at a rate referred to as the dilution rate. The dilution rate is determined by feedback from sensors in the culture to maintain a desired parameter.

A turbidostat is a continuous bioreactor in which cell density (measured, e.g., by turbidity) is kept constant. Additionally or alternatively, the culture volume of the turbidostat can be kept constant. Cell density or biomass is determined by a proxy measure such as turbidity or capacitance. Once cells reach a steady state, if cell density is maintained, the growth rate will be constant. In this system nutrient can be maintained in excess to achieve a maximum or desired growth rate. The maximal growth rates change depending on the medium formulation and process conditions. In certain embodiments, while culturing a cell culture under a turbidostat mode, the growth rate of the culture is maximized and/or set to a desired rate and, as a result, bioproduction of the culture product in limited. In certain embodiments, no culture product is generated while culturing a cell culture under a turbidostat mode. In certain embodiments, the turbidostat is coupled to a volume sensor to maintain both constant turbidity and volume by removing culture from the turbidostat once volume exceeds a set point.

A chemostat is a type of continuous bioreactor that maintains a steady-state of culture conditions and, thereby, growth rate. Culture conditions are maintained by continuously adding medium to the culture in order to maintain concentration of nutrients and other chemicals, while also removing culture liquid to keep the culture volume constant. The growth rate can be adjusted physically by changing the rate of addition of chemicals to the chemostat, and by altering rate of removal of culture liquid.

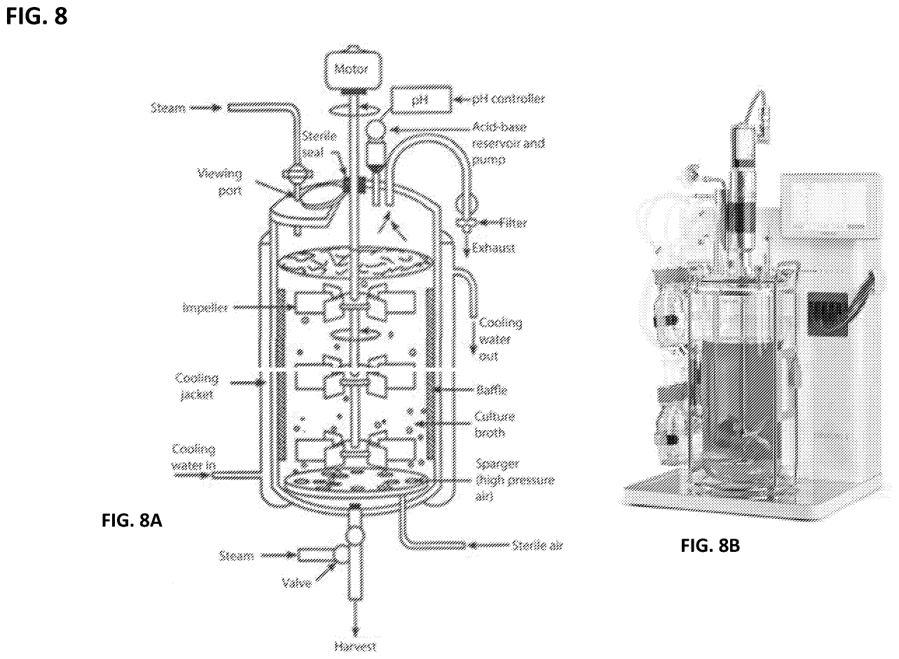

FIGS. 8A and 8B show exemplary bioreactors. FIG. 8B shows a bioreactor attached to a computer with a user interface that indicates parameters of the culture than that accepts user instructions for controlling culture conditions. Bottles comprising nutrients of various sorts are attached to the culture through tubes. Bottles may also accept excess culture medium from the culture.

Chemostats and turbidostats generally have volumes between about 500 mL and 50 L. This includes, for example, volumes between about 1 L and about 10 L. Industrial size fermenters can have sizes between about 5 liters and 500 liters.

Bioreactors are commercially available from, for example, Sartorius (Goettingen, Germany), and ThermoFisher (Waltham, MA).

### C. Culture Conditions and Culture Parameters

Culture conditions comprise a plurality of culture parameters, referred to herein as “parameter sets”, in which cells are cultured. A change in a measure of any culture parameter in a parameter set creates a new culture condition. Culture parameters include, without limitation, temperature, pH, dissolved oxygen, carbon feed rate, and nitrogen feed rate. Other culture parameters include, for example, concentration of nutrient. Compounds whose concentration can be manipulated include, for example, a metal (e.g., iron, zinc, cobalt, copper, nickel, manganese, molybdate, selenite and other transition metals), a vitamin (e.g., niacin, pyridoxine, riboflavin, pantothenate, aminobenzoic acid(s), thiamine, biotin, cyanocobalamin, folic acid), an inducer, a salt, a nitrogen sparing rate, an aeration rate, an oxygen sparging rate, a carbon dioxide sparing rate, phosphate, sulfate, chloride, acetate, citrate and other anionic salt, magnesium, calcium, sodium, potassium, ammonium and other cationic salt, boric acid, choline, ascorbic acid, lipoic acid, nicotinic acid, inositol and other vitamins, antifoaming agents (e.g., antifoam 204, antifoam A, antifoam C), amino acids (e.g., glutamate, leucine, and tryptophan), nucleic acid bases (e.g., adenine, cytosine, thymine, uracil, and guanine), complex nutrients (e.g., yeast extract, peptone, tryptone, and casamino acids), a macro-nutrient, and/or a micro-nutrient. In cell culture of eukaryotic cells, e.g., mammalian cells, culture parameters can include any of concentration of cell growth factors, CO₂ concentration, agitation speed, and antibiotic concentration.

The carbon source can be a sugar (e.g., glucose, xylose, sucrose, glycerol, or acetate), molasses, malt extract, starch, dextrin, fruit pulp, CO or CO₂. The nitrogen source can be, for example, an amino acid or polypeptide, urea, ammonium salt (e.g., ammonium sulphate, ammonium phosphate or ammonia), corn steep liquor, yeast extract, peptone, and soy bean meal.

### D. Culture Outputs

A culture output can be any measurable characteristic of a culture to be optimized. Culture outputs include, for example, cell biomass, molecular products, and proxies of product production or cell health based on cell physiology.

In certain embodiments, the culture output is the biomass of the cells themselves. In this case, the culture output to be optimized can be cell growth rate. Cell growth rate can be measured as a function of change in turbidity over time.

In other embodiments the culture output is a chemical product produced by the cells (“culture product”). The culture product can be a molecular entity that is the product of fermentation or gene expression and which, typically, is a product to be harvested from the culture and commercialized. Culture products contemplated herein include polypeptides, e.g., proteins, enzymes, antibodies. Culture products also include organic molecules that are the product of synthetic pathways in the cell, e.g., mediated by enzymes. Such products include, for example, industrial chemicals. These include, without limitation, flavorings (e.g., vanillin); flagrances (e.g., aldehydes, coumarins, indoles), amino acids, organic acids (e.g., citric, lactic and acetic acids); alcohols (e.g., ethanol, isopropanol, ketones such as acetone); fatty acids (e.g., palmitic and oleic acid). The culture output can be measured directly as concentration or as a function of amount, for example, as volumetric production rate, product titer, product yield, or specific production rate.

In other embodiments, the culture output is a proxy of output production based on, e.g., cell physiology (“culture proxy”). Culture proxies are measurable parameters that indicate health and/or physiology of a culture of cells. These include, without limitation, specific CO₂ generation rate, specific O₂ consumption rate, organic acid profile, metabolite profile, side product profile, production economics (the cost to produce 1 g of product under particular cell culture conditions). Specific rates can be defined as the rate of change of a compound per bacterium per hour, or volumetric production rate as the rate of change of a compound per liter of fermentation media per hour.

### E. Cultures of Cells

Cells to be cultured by the methods disclosed herein can be any type of cell. This includes prokaryotes and eukaryotes. Prokaryotes can include bacteria and archaea. Eukaryotes can include fungi, plants, and animals. Fungi include, for example, yeast. Plants include, for example, algae. Animal cells include those from any phylum, such as arthropods (e.g., insects, shrimp, lobster, crayfish and crabs) and chordates (e.g., fish, amphibians, reptiles, birds (e.g., chickens or turkeys) and mammals (e.g., human or non-human animals, such as bovine, lamb, goat, pig, horse, dog, cat, primate)).

Cell lines can include, for example CHO (Chinese Hamster Ovary cells), BHK21 (Baby Hamster Kidney), NS0, Sp2/0 Murine Cell lines, insect cells (e.g., SP9, Sf9, sf21, S2) tobacco BY-2 cells, Oryza Sativa and algal cells.

Cells producing a molecular product may do so constitutively or by induction. In the case of inducible system an inducer, e.g., a molecule or light if the promoter is photo inducible, can be introduced into the culture. In the case of a stress-inducible promoter, the stressor is introduced. For example, in the case of a phosphate starvation inducible promoter, phosphate can be limited or removed from the culture.

### F. Measurements

A measurement of a variable, such as a culture output, can be any combination of numbers and words. A measure can be any scale, including nominal (e.g., name or category), ordinal (e.g., hierarchical order of categories), interval (distance between members of an order), ratio (interval compared to a meaningful “0”), or a cardinal number measurement that counts the number of things in a set. Measurements of a variable on a nominal scale indicate a name or category, e.g., category into which the sequencing read is classified. Measurements of a variable on an ordinal scale produce a ranking, such as “first”, “second”, “third”. Measurements on a ratio scale include, for example, any measure on a pre-defined scale, absolute number of reads, normalized or estimated numbers, as well as statistical measurements such as frequency, mean, median, standard deviation, or quantile. Measurements that involve quantification are typically determined at the ratio scale level.

Culture outputs can be measured by any methods known in the art. For example, growth rate is measured as a function of turbidity of the culture. Proteins can be measured, for example, by immunoassay. Enzymes can be measured by enzyme assays. Small molecules can be measured by gas chromatography, HPLC or mass spectrometry.

### II. Processes

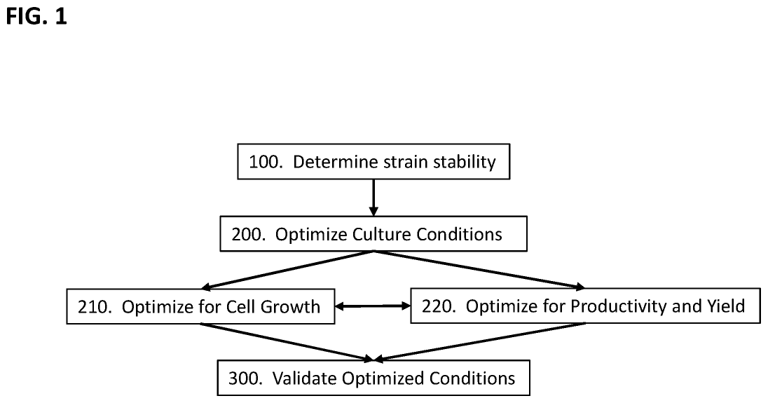

Referring to FIG. 1, optimization of culture conditions for a culture output can involve three general phases. In a first phase 100, genetic stability of the cells in culture are established. In a second phase 200, culture conditions are optimized for cell growth 210 and/or for culture output or a proxy therefore 220. In a third phase 300, culture conditions are validated at scale. Each of these three phases can be performed independently, or together.

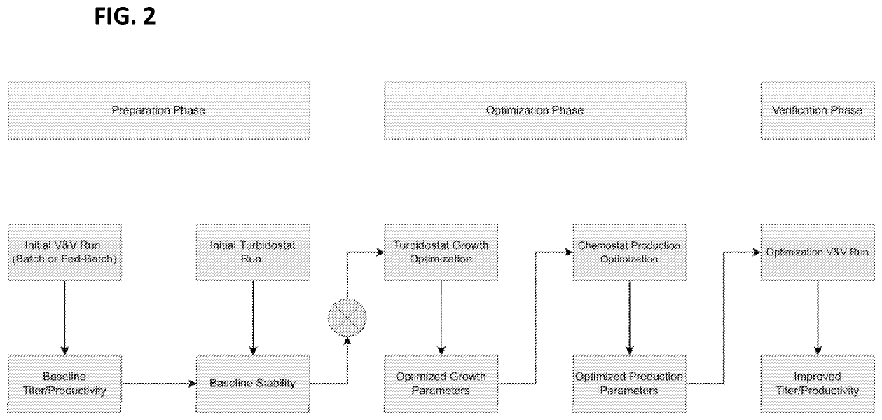

These processes are depicted in more detail in FIG. 2. A preparation phase can involve an initial validation and verification run in which baseline titer and productivity are established. In an initial turbidostat run baseline genetic stability is established. In an optimization phase, optimized growth parameters are determined in a turbidostat, and optimized production parameters are determined using a chemostat. In a verification phase, improved titer and productivity of the selected, optimized culture conditions is confirmed at scale compared to baseline in a fed-batch or batch run.

### A. Establishing Genetic Stability

A first phase of optimization can involve determining the genetic stability of cells in culture. Genetic stability refers to the number of generations cells in culture can reproduce before production of a particular culture output significantly deteriorates. It also can be represented as a period of time in culture production of a particular culture output significantly deteriorates. Production of a culture output is said to significantly deteriorate when the production of the culture output deteriorates by at least 20%, at least 25% or at least 50%.

To determine genetic stability, cells are established in continuous culture. Once established, production of a culture output is measured sequentially over time. At some point, production of the output will deteriorate. The number of generations, or time it takes for production of the output to deteriorate, which can also be measured in terms of generation cycles, represents the stability of the cell strain.

### B. Optimizing Culture Conditions

Provided herein is a process for optimizing culture conditions in a culture process for producing a culture output. The process involves determining a prior set of culture outputs for each of a set of prior culture conditions; testing new culture conditions expected to improve on the prior culture outputs to determine new culture outputs; and repeating the prior operation until a new culture condition is identified that improves upon the best prior measure of culture output. The step of identifying new culture conditions that produce improved culture outputs over prior culture conditions can be iterated to produce culture conditions exhibiting continual improvement of the culture output can be performed iteratively to continually improve culture conditions. This process can be continued until, for example, a target measure of the culture output is achieved, no further improvement in the measure of the culture output is achievable after several iterations, or a culture parameter space is exhausted or deemed to be sufficiently explored.

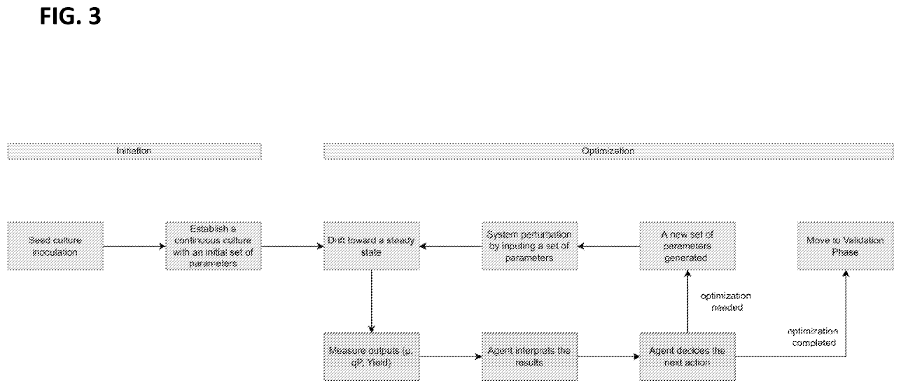

FIG. 3 shows an optimization process in more detail. A seed culture is established used to inoculate the culture and continuous culture conditions are used with an initial set of culture parameters. In an optimization phase, culture is allowed to drift toward a steady-state. One or more culture outputs are measured. An operator or an agent, such as an automated agent utilizing reinforcement learning, interprets the results and decides what parameters to change that are predicted to result in improved performance. The system is perturbed using these parameters and allowed to return to steady-state. Culture outputs are measured again. The process is iterated until optimization is complete. At this point, the operator can move to the validation phase.

### 1. Optimization

Optimization is a process in which a measured output of the process improves over a plurality of iterations in which experimental parameters are changed. The plurality of iterations can be at least any of 2, 3, 4, 5, 6, 7, 8, 9, 10, 11, 12, 13, 14, 15, 16, 17, 18, 19, 20, 25, 30, 40, 50, 60, 70, 80, 90, or 100. A process can be said to be optimized, when repeated changing of experimental parameters within a parameter space fails to improve the measure of the output. Typically, such optimization is achieved after 4-15 iterations, when the parameter space includes between 2 and 8 different parameters being tested. A goal is to identify a minimum or maximum in multidimensional (culture condition) space. Existence of local maxima or minima may imply an optimized process within a limited parameter space. However, a broader exploration of the parameter space may result in discovery of a better optimal result. Accordingly, optimization can identify a local maximum or minimum or a global maximum or minimum over the parameter space.

One or a plurality of parameters, or variables, can be tested in an optimization process. The parameters being optimized can be one or more, two or more, three or more, four or more, five or more, six or more, seven or more, or eight or more. The boundaries of any parameter are the greatest and smallest values the parameter reasonably may take. The parameter space is space bounded by boundaries of all parameters being tested. So, for example, the parameter space for pH and temperature may be that space bounded by pH=2 and pH 10.0; and temperature=15° C. and temperature=50° C. Any pair of numbers within these two ranges is said to be within the parameter space. An exhaustion of the parameter space would include testing combinations throughout the space. This could include, for example, the space near the extremes of each parameter, as well as combinations between the extremes. So, for example, if the pH extremes are pH 2 and pH 10, and the temperature extremes tested are 15° C. and 50° C., then an exhaustive search would include at least measurements at about pH 2 and about 15° C.; about pH 2 and about 50° C.; pH 10 and about 15° C.; and pH 10 and about 50° C. Inside these extremes, a plurality of combinations would also test around the mid-range, (e.g., about pH 6 and about 32° C.).

One or a plurality of culture outputs can be optimized. For example, conditions can be optimized for growth rate or for production of culture product. Alternatively, more than one culture output can be optimized simultaneously. Culture conditions can be optimized for more than one culture output by weighting the culture outputs in the decision process. For example, CO₂ production is an indication of how much nutrient is being converted into product. Therefore, lower CO₂ production rates can be better. Accordingly, both CO₂ production and product titer can be measured. An optimum might combine scores for both, weighing product titer more highly than CO₂ production rate.

### 2. Iterative Testing

Using methods described herein, culture parameter space can be quickly and efficiently explored to identify optimal culture conditions for one or more selected culture outputs.

In advance, the user selects a strain of cells to be cultured, and culture output(s), the measure of which is to be optimized. Cells are deposited in a bioreactor with culture medium, and, typically, a steady state is established in continuous culture. Desired culture outputs can be cell growth rate, production of a molecular entity, cost of production, or a proxy of these.

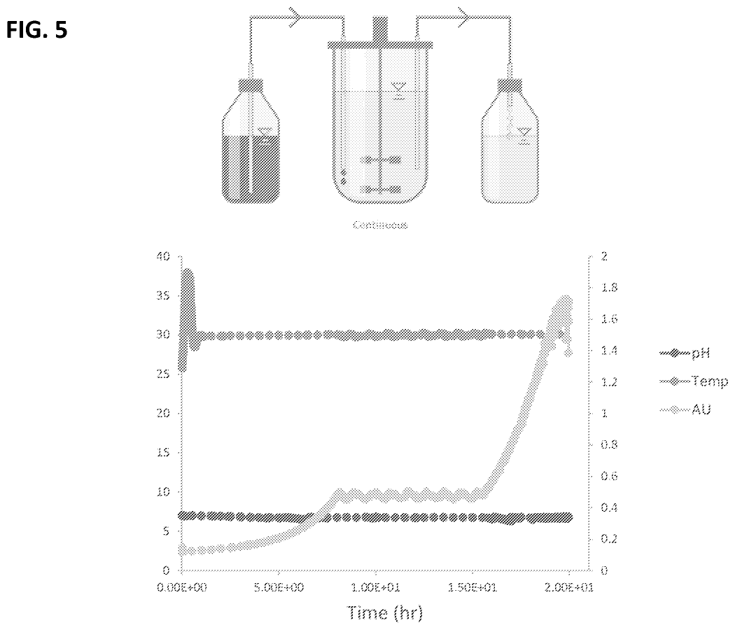

FIG. 5 shows maintenance of cells at a steady state of growth over time. Cell concentration is indicated by “AU”. When one is optimizing for cell growth, that is, when growth rate is the culture output being optimized, a turbidostat can be used, which maintains constant turbidity of the culture, which reflects cell density. Culture parameters are initially set and allowed to reach steady state. This may require, for example, 3 to 5 or more generations of cells. Certain of the culture parameters will be those who space is to be tested to identify optimal growth condition(s). The turbidostat maintains cell density by removing culture medium containing cells from the bioreactor and replacing the volume with fresh medium. Accordingly, growth rate is a function of the amount of culture medium removed over time when the culture is at steady state.

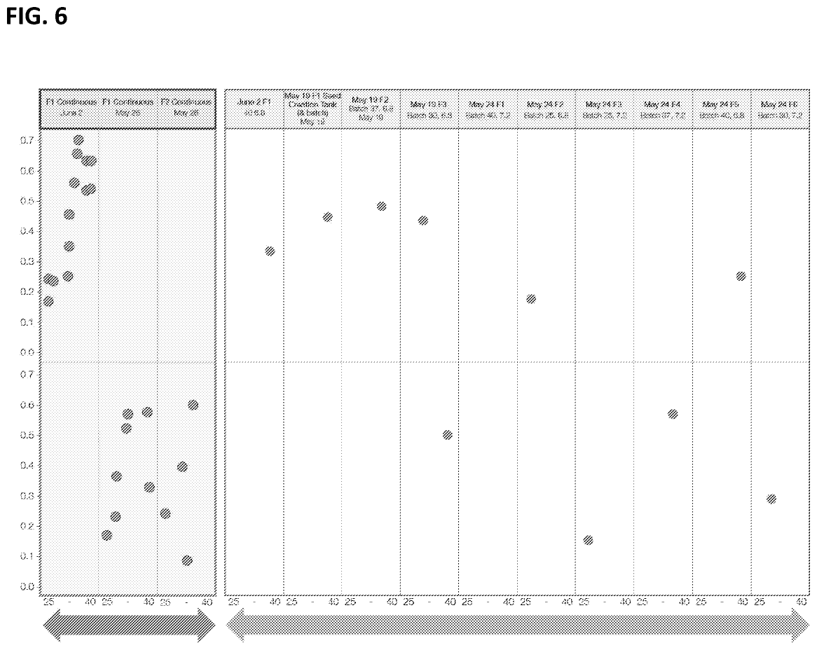

The speed of iterative testing in continuous culture is shown in FIG. 6. Up to 12 different conditions are shown tested in a single run in continuous culture. In contrast a single culture condition can be tested in one run of a batch culture.

The process of optimizing culture conditions is iterative. It involves beginning with initial measures of the culture output. Rate of cell growth might be given, for example, as the doubling time of cells in the culture. Rate of cell growth might be initially tested under a plurality of different culture conditions. Changing culture conditions can involve changes to only one variable at a time, such as changes in temperature or in pH, or it can involve changes in a plurality of the variables, such as changing both temperature and pH. In any case, initial measures of the culture output under one or more different conditions function as “priors” on which future culture conditions can be selected.

A human operator or an intelligent agent now selects new culture conditions for testing. Typically, the selected new culture conditions are predicted by the operator or agent to improve the measure of the culture output, and are based on one or more prior measures of the culture output. So, for example, if three different culture conditions comprising increases in culture temperature produce increases in culture output, the operator or agent might predict that a further increase in temperature would also produce a further increase culture output. Accordingly, the operator or agent may select a new culture condition in which temperature is increased further. The culture is then perturbed to reflect the new culture condition and the culture output is once again measured.

The new measure can be taken once the culture has reached steady state. Alternatively, the measure of culture output can be based on tests of the culture after a certain time period or using a plurality of measurements over time to identify a trend, which trend may be used to predict an ultimate measure of culture output.

This process, which involves checking prior measures of culture output, predicting changes in culture conditions that will improve culture output based on these priors, perturbing the system with the predicted culture conditions and measuring culture output of the perturbed system, is iterated. The number of iterations can continue until a particular event is achieved. This could be until the genetic stability of the strain is reached. That is, iterations can be tested for the number of generations until product output begins to deteriorate on account of the genetic instability of the cells. Iterations can continue until a target culture output is achieved. For example this could be a certain rate of molecular product production. One indication that culture conditions is optimized is that continued altering of culture conditions fails to produce any further improvement in culture output. It is to be understood that the existence of local maxima or minima within the culture parameter space may provide a false indication of global optimization. Therefore, different points across the parameter space can be tested so as not to be trapped in local min/max areas.

An initial set of conditions for testing for culture product can be informed by those culture conditions found to be optimized for rate of cell growth. For example the initial parameters set can be the same as or about the same as those identified at the cell growth stage. Initial culture parameters are about the same as previous parameters if each culture parameter is about the same as the previous parameter, that is, within plus or +/−5% of the measure of the previous culture parameter.

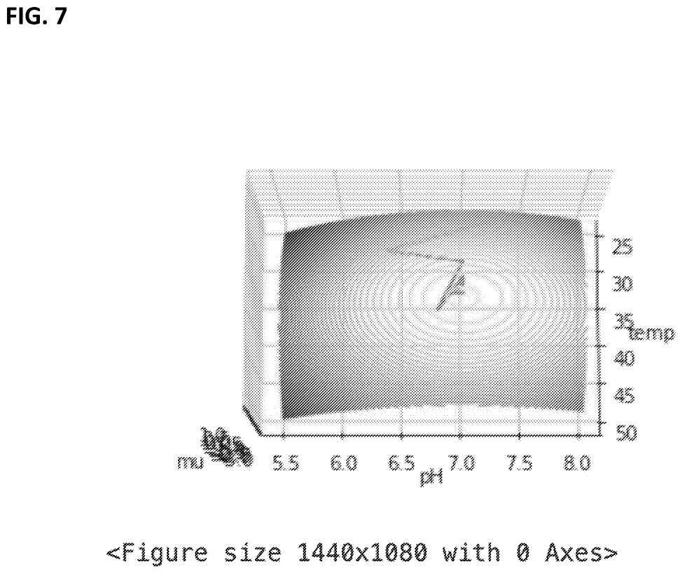

FIG. 7 shows an exemplary set of iterations testing two variables. In this case, the parameters to be varied are pH and temperature. The culture output, mu, represents growth rate of the organism. After several trials, optimum values for the parameters that maximize mu are identified at about pH 7 and temperature about 37° C.

It is also understood that this process can include testing of new culture conditions that are not necessarily predicted to improve the culture output, in order to provide more data to include in the priors. Such testing can occur at any point in the iterative process. Testing of different culture conditions can involve a methodical process of systematically changing variables for each culture parameter across the parameter space. Such a process would identify culture condition(s) producing an optimum for the culture output. However, such process is likely to be slower and less efficient then an intelligent process in which prior results are used to predict culture conditions that improve culture output.

Accordingly, this process can be used to identify culture conditions producing the greatest growth rate for the culture of cells. Additionally, the process can be used to identify optimal culture conditions for production of a product, or culture conditions that minimize the cost of producing a product.

In certain embodiments the culture output measured can be the amount of a particular molecular product produced. In other embodiments the absolute amount of product produced may be less important than the economics of product production. So, for example, the culture output may be the relative cost to produce a unit of the molecular product. This, in turn, can take into account the cost of all the inputs in the value of the output. This would reflect the value add of the process.

### C. Validating Culture Conditions at Scale

After optimal conditions are identified through the iterative testing method in continuous culture, described herein, these conditions can be validated at scale. Typically, production of product industrial systems is performed in (fed)-batch reactors at scales of around 10,000 liters to 1,000,000 liters. The validation step involves establishing a culture of cells in a (fed)-batch reactor under the selected optimal culture conditions and measuring the culture output relative to the baseline starting conditions. If culture output in in a (fed)-batch reactor, proves to correlate reasonably with output under the determined optimal conditions in continuous culture, these conditions can be used in commercial processes for producing the product.

### III. Systems

Also provided herein are systems for optimizing culture conditions for the production of a culture output. Such systems comprise a bioreactor for growing cells and the computer comprising an intelligent agent, and programmed to instruct the bioreactor to establish culture conditions and to predict culture conditions expected to improve culture outputs from prior data.

### A. Computer Systems

Models provided herein can be executed by programmable digital computer.

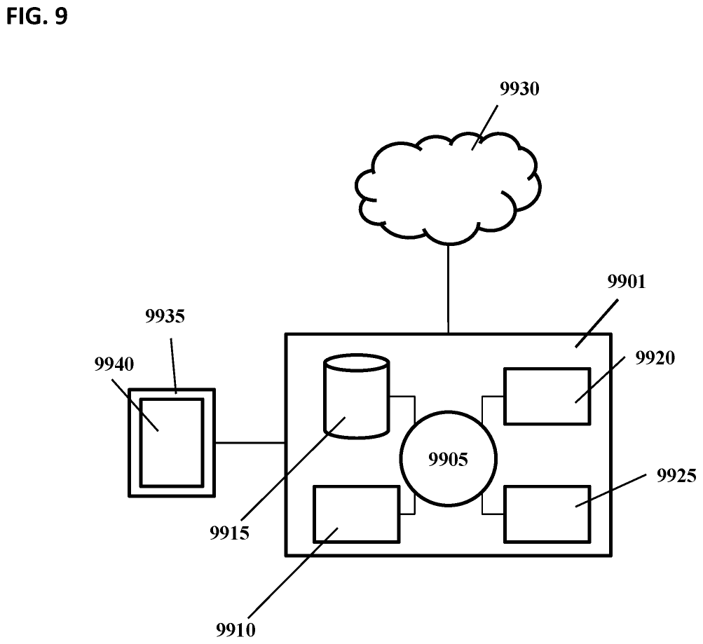

FIG. 9 shows an exemplary computer system. The computer system 9901 includes a central processing unit (CPU, also “processor” and “computer processor” herein) 9905, which can be a single core or multi core processor, or a plurality of processors for parallel processing. The computer system 9901 also includes memory or memory location 9910 (e.g., random-access memory, read-only memory, flash memory), electronic storage unit 9915 (e.g., hard disk), communication interface 9920 (e.g., network adapter) for communicating with one or more other systems, and peripheral devices 9925, such as cache, other memory, data storage, bioreactors, and/or electronic display adapters. The computer readable memory 9910, storage unit 9915, interface 9920 and peripheral devices 9925 are in communication with the CPU 9905 through a communication bus (solid lines), such as a motherboard. The storage unit 9915 can be a data storage unit (or data repository) for storing data. The computer system 9901 can be operatively coupled to a computer network (“network”) 9930 with the aid of the communication interface 9920. The network 9930 can be the Internet, an internet and/or extranet, or an intranet and/or extranet that is in communication with the Internet. The network 9930 in some cases is a telecommunication and/or data network. The network 9930 can include one or more computer servers, which can enable distributed computing, such as cloud computing.

The CPU 9905 can execute a sequence of machine-readable instructions, which can be embodied in a program or software (code). The instructions may be stored in a memory location, such as the computer readable memory 9910. The instructions can be directed to the CPU 9905, which can subsequently program or otherwise configure the CPU 9905 to implement methods of the present disclosure.

The storage unit 9915 can store files, such as drivers, libraries, and saved programs. The storage unit 9915 can store user data, e.g., user preferences, log files, video or other images, and user programs. The computer system 9901 in some cases can include one or more additional data storage units that are external to the computer system 9901, such as located on a remote server that is in communication with the computer system 9901 through an intranet or the Internet.

The computer system 9901 can communicate with one or more remote computer systems through the network 9930.

Methods as described herein can be implemented by way of machine (e.g., computer processor) executable code stored on an electronic storage location of the computer system 9901, such as, for example, on the computer readable memory 9910 or electronic storage unit 9915. The machine executable or machine-readable code can be provided in the form of software. During use, the code can be executed by the processor 9905. In some cases, the code can be retrieved from the storage unit 9915 and stored on the memory 9910 for ready access by the processor 9905. In some situations, the electronic storage unit 9915 can be precluded, and machine-executable instructions are stored on memory 9910. The code can be used to communicate and issue instructions to electronic devices, e.g., circuit boards 9940, modules, or subsystems, on the instrument.

The computer system 9901 can communicate with one or more remote computer systems through the network 9930.

Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks.

The computer system 9901 can include or be in communication with an electronic display 9935 that comprises a user interface (UI) 9940 for providing, for example, input parameters for methods described herein. Examples of UIs include, without limitation, a graphical user interface (GUI) and web-based user interface.

Further provided herein are computer systems comprising software products in tangible form comprising code that, when executed, directs systems provided herein to perform optimization methods described herein.

### Machine Learning Optimization

Machine learning optimization is the process of iteratively improving the accuracy of a machine learning model, lowering the degree of error. Optimization is measured through a loss or cost function, which is typically a way of defining the difference between the predicted and actual value of data. Machine learning models aim to minimize this loss function. In the current case, the cost function can be the inverse of production level of a culture output. Accordingly, minimizing the cost function is equivalent to maximizing production of the culture output. Machine learning optimization methods include, for example, random search, gradient descent, and genetic algorithm.

Random search, or exhaustive search, is a method of testing points throughout the parameter space, and identifying a minimum of the cost function based on these results.

In gradient descent, an arbitrary value for a parameter or set of parameters is chosen and tested. Then, a nearby value for the parameter in one direction (e.g., higher or lower) is tested and determined to be closer or farther away from the minimum. If farther away, the next parameter value will be selected in the other direction and tested. This process can continue until a local or global minimum is found.

Genetic or evolutionary algorithms test a plurality of sets of parameter values, and choose values for the parameters that minimize the cost function. So, for example, different combinations of pH, temperature, and dissolved oxygen are tested for culture output. Values on each parameter that are associated with improved culture output are selected, recombined and tested again to identify yet better combinations of values for the parameters.

One deep learning technique is reinforcement learning. Reinforcement learning is an aspect of machine learning where an agent learns to behave in an environment by performing certain actions in observing the rewards/results which it gets from those actions. A schematic for reinforcement learning is depicted in FIG. 3. An agent takes an action (aₜ) on its environment. This produces information about the environment state (Sₜ) and a reward (Rₜ) indicating whether the result is better than the previous result. The agent works on the hypothesis of reward maximization.

One version of reinforcement learning is referred to as direct search. “Direct search” refers to sequential examination of trial solutions involving comparison of each trial solution with the “best” obtained up to that time, together with a strategy for determining (as a function of earlier results) what the next trial solution will be.

Q-learning is a model-free reinforcement learning algorithm to learn the value of an action in a particular state.

In some variations, analysis may involve implementing machine learning techniques including linear and non-linear models, e.g., processes such as CART—classification and regression trees), artificial neural networks such as back propagation networks, discriminant analyses (e.g., Bayesian classifier or Fischer analysis), logistic classifiers, and support vector classifiers (e.g., support vector machines).

Machine learning algorithms for identifying optimal culture conditions can take advantage of deep learning techniques. Deep learning techniques make use of multiple layers in the learning process.

Artificial neural networks use collections of interconnected nodes. Neural networks are comprised of a node layers, containing an input layer, one or more hidden layers, and an output layer. Each node, or artificial neuron, connects to another and has an associated weight and threshold. If the output of any individual node is above the specified threshold value, that node is activated, sending data to the next layer of the network. comprised of a node layers, containing an input layer, one or more hidden layers, and an output layer. Each node, or artificial neuron, connects to another and has an associated weight and threshold. If the output of any individual node is above the specified threshold value, that node is activated, sending data to the next layer of the network

### EXEMPLARY EMBODIMENTS

1. A method for iteratively improving culture conditions for at least one culture output in a culture process, comprising:
a) providing a continuous culture of cells in a bioreactor, wherein the cells are cultured under culture conditions that comprise a parameter set of culture parameters, and wherein the culture of cells produces at least one culture output at a first measure;
b) while maintaining the cells in continuous culture, iteratively testing different culture conditions by:
   (i) selecting a new culture condition comprising a new parameter set; wherein the new culture condition is predicted to improve the measure of the culture output, and is selected based on one or more prior measures of the culture output;
   (ii) perturbing the cells with the new culture conditions; and
   (iii) determining a new measure of the culture output;
   wherein testing is iterated to produce a plurality of improvements in the measure of culture output.

2. The method of embodiment 1, comprising iteratively testing different culture conditions until:
(1) a target output measure is achieved; or
(2) a plurality of iterations fails to identify a culture condition that improves on a prior optimal output measure.

3. The method of embodiment 1, further comprising testing new culture conditions until a search of user defined parameter space is exhausted.

4. The method of embodiment 1, wherein selecting is performed using an automated agent.

5. The method of embodiment 4, wherein the automated agent uses an artificial intelligence method.

6. The method of embodiment 5, wherein the artificial intelligence method is selected from reinforcement learning, deep learning, Q-learning, and neural net.

7. The method of embodiment 4, wherein the automated agent uses a direct search method (e.g., the Nelder-Mead method, a dynamic programming method, a downhill simplex method, a simplex algorithm, linear regression, binary search tree, or random walk).

8. The method of embodiment 1, further comprising, before operation (b): determining baseline genetic stability of the cells in culture.

9. The method of embodiment 1, comprising iterating operation (b) at least any of 2, 3, 4, 5, 10, 15, 20, 25, 30, 50 or 100 (e.g., between 4 and 15) iterations.

10. The method of embodiment 1, wherein the culture output comprises a weighted score comprising a plurality of different culture outputs.

11. The method of embodiment 1, further comprising:
c) choosing from among the parameter sets, a parameter set that optimizes the measure of the culture output; growing cells in a batch culture (e.g., fed-batch culture) with the chosen parameter set; and measuring the culture output.

12. The method of embodiment 1, wherein the volume of the culture is between about 50 ml and about 15 L, e.g., between about 500 mL and about 10 L.

13. The method of embodiment 1, wherein a culture output is specific growth rate.

14. The method of embodiment 1, wherein a culture output is selected from product titer, product yield, specific production rate, volumetric production rate.

15. The method of embodiment 1, wherein a culture output is selected from biomass yield, specific CO₂ generation rate, specific O₂ consumption rate, organic acid profile, metabolite profile, side product profile, and production economics.

16. The method of embodiment 1, wherein a culture output is selected from a polypeptide (e.g., proteins, enzymes, antibodies), an organic molecule that is the product of a synthetic pathway in the cell (e.g., an industrial chemical such as a flavoring (e.g., vanillin); a flagrance (e.g., aldehydes, coumarins, indoles), an amino acid, an organic acid (e.g., citric, lactic and acetic acids); an alcohol (e.g., ethanol, isopropanol, ketones such as acetone); and a fatty acid (e.g., palmitic and oleic acid).

17. The method of embodiment 13, wherein specific rates are defined as the rate of change of the culture output per bacterium per hour.

18. The method of embodiment 1, wherein the cells comprise archaea, prokaryotes and/or eukaryotes.

19. The method of embodiment 1, wherein the cells comprise fungal cells (e.g., yeast (e.g., Saccharomyces cerevisiae, Pichia spp., Kuyveromyces spp or Aspergillus spp, Rhodoporidium spp Lipolytica spp, Aspergillus spp, Neurospora spp Trichoderma spp, Candida spp, Penicillium).

20. The method of embodiment 1, wherein the cells comprise bacterial cells (e.g., Escherichia coli, Bacillus spps, Costridia spp, Streptomyces spp, Pseudomonas spp, Ralstonia spp, Shewanella spp,).

21. The method of embodiment 1, wherein the cells comprise insect cells, animal cells or plant cells.

22. The method of embodiment 1, wherein the cells comprise animal cells (e.g., arthropods (e.g., insects, shrimp, lobster, crayfish and crabs) and chordates (e.g., fish, amphibians, reptiles, birds (e.g., chickens or turkeys) and mammals (e.g., human or non-human such as bovine, lamb, goat, pig, horse, dog, cat, primate)).

23. The method of embodiment 1, wherein the cells comprise a cell line (e.g., CHO (Chinese Hamster Ovary cells), BHK21 (Baby Hamster Kidney), NS0, Sp2/0 Murine Cell lines, insect cells (e.g., SP9, Sf9, sf21, S2) tobacco BY-2 cells, Oryza Sativa and algal cells.

24. The method of embodiment 1, wherein the measure of culture output is based on cells grown to steady state.

25. The method of embodiment 1, wherein the measure of culture output is based on a trend line of cells during growth.

26. The method of embodiment 1, wherein the measure is made between 1 and 24 hours after perturbing.

27. The method of embodiment 1, wherein culturing is performed in a turbidostat or a chemostat.

28. The method of embodiment 1, wherein one or more of the culture parameters comprise one or more of temperature, pH, dissolved oxygen, carbon feed rate, and nitrogen feed rate.

29. The method of embodiment 1, wherein one or more culture parameters comprise concentration of nutrient.

30. The method of embodiment 1, wherein one or more culture parameters comprise concentration of a metal (e.g., iron, zinc, cobalt, copper, nickel, manganese, molybdate, selenite and other transition metals), a vitamin (e.g., niacin, pyridoxine, riboflavin, pantothenate, aminobenzoic acid(s), thiamine, biotin, cyanocobalamin, folic acid), an inducer, a salt, a nitrogen sparing rate, an aeration rate, an oxygen sparging rate, a carbon dioxide sparing rate, phosphate, sulfate, chloride, acetate, citrate and other anionic salt, magnesium, calcium, sodium, potassium, ammonium and other cationic salt, boric acid, choline, ascorbic acid, lipoic acid, nicotinic acid, inositol and other vitamins, antifoaming agents (e.g., antifoam 204, antifoam A, antifoam C), amino acids (e.g., glutamate, leucine, and tryptophan), nucleic acid bases (e.g., adenine, cytosine, thymine, uracil, and guanine), complex nutrients (e.g., yeast extract, peptone, tryptone, and casamino acids), a macro-nutrient, and/or a micro-nutrient.

31. The method of embodiment 1, wherein one or more culture parameters comprise concentration of a cell growth factor, concentration of CO₂, culture agitation speed, and concentration of an antibiotic.

32. The method of embodiment 1, wherein a culture parameter comprises concentration of a carbon source.

33. The method of embodiment 32, wherein the carbon source is selected from a sugar (e.g., glucose, xylose, sucrose, glycerol, or acetate), molasses, malt extract, starch, dextrin, fruit pulp, CO or CO₂.

34. The method of embodiment 1, wherein a culture parameter comprises concentration of a nitrogen source.

35. The method of embodiment 34, wherein the nitrogen source is selected from an amino acid or polypeptide, urea, ammonium salt (e.g., ammonium sulphate, ammonium phosphate or ammonia), corn steep liquor, yeast extract, peptone, and soy bean meal.

36. The method of embodiment 1, wherein the parameter set comprises at least any of 1, 2, 3, 4, 5, 6, 7, or 8 (e.g., between 2 and 6) culture parameters.

37. The method of embodiment 1, comprising performing between 2 and 20 (e.g., between 8 and 15) iterations in no more than 24 hours.

38. The method of embodiment 1, comprising inducing activity of a biochemical pathway that produces a target product.

39. The method of embodiment 1, wherein the cells comprise a constitutive or inducible system for gene expression.

40. The method of embodiment 39, wherein the gene encodes a commercial product.

41. The method of embodiment 39, wherein the gene encodes an enzyme in a biochemical pathway of production of a commercial product.

42. The method of embodiment 1, wherein the product is selected from a recombinant protein, a native protein (e.g., an enzyme), a nutritional supplement, a cannabinoid, a metabolite, an organic acid(s), a lipid(s), a spore, or cellular biomass.

43. The method of embodiment 1, further comprising, before step (b):
determining baseline fermentation process of the microbial growth, production titer, and productivity in a batch or fed-batch reactor.

44. The method of embodiment 1, comprising first and second optimization process, wherein a first optimization process identifies an optimized parameter set for cell growth and a second optimization process identifies an optimized parameter set for a molecular product.

45. The method of embodiment 44, wherein the optimized parameter set for cell growth informs initial culture conditions for optimizing for the molecular product.

46. The method of embodiment 1, wherein initial parameter sets comprise randomized, repeated or selected parameter sets.

47. A system comprising:
a) a vessel configured for continuous culture of cells (e.g., a turbidostat or a chemostat);
b) one or more sensors that (i) measure one or more culture outputs of cells being cultured in the vessel, and (ii) transmit the measurements to computer memory;
c) one or more feeds that feed compounds (e.g., nutrients) into the vessel;
d) a computer comprising: (i) a processor; and (ii) a memory, coupled to the processor, and comprising a module comprising:
   (1) a dataset comprising, for each of a plurality of culture conditions: measures of culture parameters and measures of culture outputs received from the sensors and associated with the culture conditions;
   (2) an automated agent comprising an algorithm that selects a new culture condition predicted to produce an improved measure in the culture output and based on the dataset; and
   (3) computer executable instructions that implement the new culture condition in the system, measure the culture output, and add the culture condition and the culture output to the dataset.

48. The system of embodiment 47, wherein the automated agent uses an artificial intelligence method.

49. The system of embodiment 48, wherein the artificial intelligence method is selected from reinforcement learning, deep learning, Q-learning, and neural net.

50. The system of embodiment 47, wherein the automated agent uses a directed search method (e.g., the Nelder-Mead method, a dynamic programming method, a downhill simplex method, a simplex algorithm, linear regression, binary search tree, or random walk).

51. The system of embodiment 47, comprising one or more of:
A) mixer (e.g., an impeller or a pneumatic agitator) to mix liquid in the vessel, and actuated by a motor;
B) a temperature regulator and a temperature sensor;
C) a source of acidic and alkaline reagents communicating with the vessel interior and a PH meter;
D) an aeration system communicating with the vessel interior and a dissolved oxygen meter;
E) a source of nutrients communicating with the vessel interior and an analyzer for measuring the nutrient;
F) an effluent communicating with the vessel interior and a regulatable valve or pump for regulating fluid flow from the vessel;
G) baffles in the vessel;
H) a sparger and mass flow controller communicating with the vessel interior for input of one or more gases and mixtures thereof; and
I) a user interface for communicating instructions with the computer.

52. The system of embodiment 51, wherein operation of the mixer, the temperature regulator, the source of acidic and alkaline reagents, the source of oxygen and the source of nutrients are under control of the computer.

53. A method comprising:
a) in continuous culture, determining genetic stability of cells in culture;
b) optimizing culture conditions, wherein the culture conditions comprise a parameter set of culture parameters, for a culture output by:
   (i) optimizing the culture conditions for cell growth; and/or
   (ii) optimizing the culture conditions for the product output;
   wherein optimizing comprises performing a set of operations comprising:
      I) providing initial measures of cell growth or product output under a plurality of different parameter sets;
      II) iteratively performing:
         A) based on measures of cell growth or product output, selecting a new parameter set predicted to improve cell growth or product output;
         B) perturbing the cells in culture with the new parameter set; and
         C) measuring cell growth or product output after perturbing the cells; and
      III) selecting an optimized parameter set; and
c) validating the optimized parameter set by:
   i) growing the cells in continuous or fed-batch mode under the optimized parameter set; and
   ii) measuring the culture output.

54. The method of embodiment 53, wherein selecting is performed with an automated agent.

55. The method of embodiment 53, wherein genetic stability is measured as a function of a number of generations until a measure of cell growth or product output significantly deteriorates.

56. The method of embodiment 53, wherein optimizing for cell growth is performed in a turbidostat and optimizing for culture output is performed in a chemostat.

57. The method of embodiment 53, wherein optimizing in continuous culture is performed no longer than a period of genetic stability.

58. The method of embodiment 53, wherein validating is performed at a volume of about 5 liters to about 500 liters.

59. A method comprising:
a) providing a continuous culture of cells in a bioreactor, wherein the culture of cells produces at least one culture output; and
b) while maintaining the cells in continuous culture, using a machine learning optimization method to iteratively alter and test different culture conditions along one or a plurality of different culture parameters for the production of the at least one culture output, wherein a plurality of the iterations alter culture conditions in a direction predicted by the model to improve production of the at least one culture output, wherein the iterations are continued to produce a plurality of improvements.

60. The method of embodiment 59, wherein the iterations continue until a local maximum of production is reached.

61. The method of embodiment 59, wherein the iterations are performed between 2 and 25 times, e.g., between 5 and twenty times.

62. The method of embodiment 59, wherein the iterations are performed at least 5, 6, 7, 8, 9, 10, 11, 12, 13, 14, 15, 16, 17, 18, 19, 20, 21, 22, 23, 24 or 25 times.

63. The method of embodiment 59, wherein the iterations are performed to produce at least any of 5, 6, 7, 8, 9, 10, 11, 12, 13, 14, 15, 16, 17, 18, 19, 20, 21, 22, 23, 24 or 25 improvements.

64. The method of embodiment 59, wherein the machine learning optimization method uses reinforcement learning, gradient descent, a genetic algorithm or random search.

65. The method of embodiment 59, wherein a plurality of iterations comprise altering culture conditions along a plurality of culture parameters.

66. The method of embodiment 65, wherein the plurality of culture parameters include one or more of pH, temperature, dissolved oxygen, carbon feed rate, and nitrogen feed rate.

67. The method of embodiment 59, further comprising culturing cells under the improved or optimized culture conditions to produce the culture product, and collecting the culture product from the culture.

68. The method of embodiment 67, wherein the culture product is produced at a scale of at least any of 500 liters, 1000 liters, 5000 liters, 10,000 liters, 50,000 liters, 100,000 liters, 500,000 liters or 1,000,000 liters.

### EXAMPLES

A continuous turbidostat culture is set up with Escherichia coli cells with a defined medium to optimize for the optimal specific growth rate (mu). The two parameters of interest are growth temperature and pH. The growth temperature and pH ranges (25°-50° C., 5.5-8.5) are pre-defined before the initiation of the optimization. The reactor randomly initialized at temp=25° C., pH=7.5, entering a steady-state condition with constant biomass concentration (turbidostat).

After three-volume replacement (i.e., three liters of medium consumed through a turbidostat with a constant one-liter volume), a machine learning or numerical search method, such as reinforcement learning or Nelder-Mead method, is implemented to search for the optimal point. After the initiation condition, the reactor changed its condition to temp=27° C., pH=6.4, then temp=29° C., pH=7.0, which each change resulted in a higher specific growth rate. Specific growth rates are measured at steady states, which are achieved at each of these conditions after three-volume replacement. Over time the reactor stops at the final optimized condition of temp=33° C., pH=7.0, the final optimized growth condition giving the highest growth rate. Exemplary results are shown in FIG. 7.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “about” refers to a range that is 5% plus or minus from a stated numerical value within the context of the particular usage. So, for example, “about 100” means between 95 and 105. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims.

All publications, patents, and patent applications mentioned in this specification are herein incorporated by reference to the same extent as if each individual publication, patent, or patent application was specifically and individually indicated to be incorporated by reference.

What is claimed is:

1. A method for iteratively improving culture conditions for at least one culture output in a culture process, comprising:
a) providing a continuous culture of cells in a bioreactor, wherein the cells are cultured under culture conditions that comprise a parameter set of culture parameters, and wherein the culture of cells produces at least one culture output at a first measure;
b) while maintaining the cells in continuous culture, iteratively testing different culture conditions by:
(i) selecting a new culture condition comprising a new parameter set; wherein the new culture condition is predicted to improve the measure of the culture output, and is selected based on one or more prior measures of the culture output;
(ii) perturbing the cells with the new culture conditions; and
(iii) determining a new measure of the culture output;
wherein testing is iterated to produce a plurality of improvements in the measure of culture output.

2. The method of claim 1, comprising iteratively testing different culture conditions until:
(1) a target output measure is achieved; or
(2) a plurality of iterations fails to identify a culture condition that improves on a prior optimal output measure.

3. The method of claim 1, further comprising testing new culture conditions until a search of user defined parameter space is exhausted.

4. The method of claim 1, wherein selecting is performed using an automated agent.

5. The method of claim 4, wherein the automated agent uses an artificial intelligence method.

6. The method of claim 5, wherein the artificial intelligence method is selected from reinforcement learning, deep learning, Q-learning, and neural net.

7. The method of claim 4, wherein the automated agent uses a direct search method (e.g., the Nelder-Mead method, a dynamic programming method, a downhill simplex method, a simplex algorithm, linear regression, binary search tree, or random walk).

8. The method of claim 1, further comprising, before operation (b):
determining baseline genetic stability of the cells in culture.

9. The method of claim 1, comprising iterating operation (b) at least any of 2, 3, 4, 5, 10, 15, 20, 25, 30, 50 or 100 (e.g., between 4 and 15) iterations.

10. The method of claim 1, wherein the culture output comprises a weighted score comprising a plurality of different culture outputs.

11. The method of claim 1, further comprising:
c) choosing from among the parameter sets, a parameter set that optimizes the measure of the culture output; growing cells in a batch culture (e.g., fed-batch culture) with the chosen parameter set; and measuring the culture output.

12. The method of claim 1, wherein the volume of the culture is between about 50 ml and about 15 L, e.g., between about 500 mL and about 10 L.

13. The method of claim 1, wherein a culture output is specific growth rate.

14. The method of claim 1, wherein a culture output is selected from product titer, product yield, specific production rate, volumetric production rate.

15. The method of claim 1, wherein a culture output is selected from biomass yield, specific CO₂ generation rate, specific O₂ consumption rate, organic acid profile, metabolite profile, side product profile, and production economics.

16. The method of claim 1, wherein a culture output is selected from a polypeptide (e.g., proteins, enzymes, antibodies), an organic molecule that is the product of a synthetic pathway in the cell (e.g., an industrial chemical such as a flavoring (e.g., vanillin); a flagrance (e.g., aldehydes, coumarins, indoles), an amino acid, an organic acid (e.g., citric, lactic and acetic acids); an alcohol (e.g., ethanol, isopropanol, ketones such as acetone); and a fatty acid (e.g., palmitic and oleic acid).

17. The method of claim 13, wherein specific rates are defined as the rate of change of the culture output per bacterium per hour.

18. The method of claim 1, wherein the cells comprise archaea, prokaryotes and/or eukaryotes.

19. The method of claim 1, wherein the cells comprise fungal cells (e.g., yeast (e.g., Saccharomyces cerevisiae, Pichia spp., Kuyveromyces spp or Aspergillus spp, Rhodoporidium spp Lipolytica spp, Aspergillus spp, Neurospora spp Trichoderma spp, Candida spp, Penicillium).

20. The method of claim 1, wherein the cells comprise bacterial cells (e.g., Escherichia coli, Bacillus spps, Costridia spp, Streptomyces spp, Pseudomonas spp, Ralstonia spp, Shewanella spp,).

21. The method of claim 1, wherein the cells comprise insect cells, animal cells or plant cells.

22. The method of claim 1, wherein the cells comprise animal cells (e.g., arthropods (e.g., insects, shrimp, lobster, crayfish and crabs) and chordates (e.g., fish, amphibians, reptiles, birds (e.g., chickens or turkeys) and mammals (e.g., human or non-human such as bovine, lamb, goat, pig, horse, dog, cat, primate)).

23. The method of claim 1, wherein the cells comprise a cell line (e.g., CHO (Chinese Hamster Ovary cells), BHK21 (Baby Hamster Kidney), NS0, Sp2/0 Murine Cell lines, insect cells (e.g., SP9, Sf9, sf21, S2) tobacco BY-2 cells, Oryza Sativa and algal cells.

24. The method of claim 1, wherein the measure of culture output is based on cells grown to steady state.

25. The method of claim 1, wherein the measure of culture output is based on a trend line of cells during growth.

26. The method of claim 1, wherein the measure is made between 1 and 24 hours after perturbing.

27. The method of claim 1, wherein culturing is performed in a turbidostat or a chemostat.

28. The method of claim 1, wherein one or more of the culture parameters comprise one or more of temperature, pH, dissolved oxygen, carbon feed rate, and nitrogen feed rate.

29. The method of claim 1, wherein one or more culture parameters comprise concentration of nutrient.

30. The method of claim 1, wherein one or more culture parameters comprise concentration of a metal (e.g., iron, zinc, cobalt, copper, nickel, manganese, molybdate, selenite and other transition metals), a vitamin (e.g., niacin, pyridoxine, riboflavin, pantothenate, aminobenzoic acid(s), thiamine, biotin, cyanocobalamin, folic acid), an inducer, a salt, a nitrogen sparing rate, an aeration rate, an oxygen sparging rate, a carbon dioxide sparing rate, phosphate, sulfate, chloride, acetate, citrate and other anionic salt, magnesium, calcium, sodium, potassium, ammonium and other cationic salt, boric acid, choline, ascorbic acid, lipoic acid, nicotinic acid, inositol and other vitamins, antifoaming agents (e.g., antifoam 204, antifoam A, antifoam C), amino acids (e.g., glutamate, leucine, and tryptophan), nucleic acid bases (e.g., adenine, cytosine, thymine, uracil, and guanine), complex nutrients (e.g., yeast extract, peptone, tryptone, and casamino acids), a macro-nutrient, and/or a micro-nutrient.

31. The method of claim 1, wherein one or more culture parameters comprise concentration of a cell growth factor, concentration of CO₂, culture agitation speed, and concentration of an antibiotic.

32. The method of claim 1, wherein a culture parameter comprises concentration of a carbon source.

33. The method of claim 32, wherein the carbon source is selected from a sugar (e.g., glucose, xylose, sucrose, glycerol, or acetate), molasses, malt extract, starch, dextrin, fruit pulp, CO or CO₂.

34. The method of claim 1, wherein a culture parameter comprises concentration of a nitrogen source.

35. The method of claim 34, wherein the nitrogen source is selected from an amino acid or polypeptide, urea, ammonium salt (e.g., ammonium sulphate, ammonium phosphate or ammonia), corn steep liquor, yeast extract, peptone, and soy bean meal.

36. The method of claim 1, wherein the parameter set comprises at least any of 1, 2, 3, 4, 5, 6, 7, or 8 (e.g., between 2 and 6) culture parameters.

37. The method of claim 1, comprising performing between 2 and 20 (e.g., between 8 and 15) iterations in no more than 24 hours.

38. The method of claim 1, comprising inducing activity of a biochemical pathway that produces a target product.

39. The method of claim 1, wherein the cells comprise a constitutive or inducible system for gene expression.

40. The method of claim 39, wherein the gene encodes a commercial product.

41. The method of claim 39, wherein the gene encodes an enzyme in a biochemical pathway of production of a commercial product.

42. The method of claim 1, wherein the product is selected from a recombinant protein, a native protein (e.g., an enzyme), a nutritional supplement, a cannabinoid, a metabolite, an organic acid(s), a lipid(s), a spore, or cellular biomass.

43. The method of claim 1, further comprising, before step (b):
determining baseline fermentation process of the microbial growth, production titer, and productivity in a batch or fed-batch reactor.

44. The method of claim 1, comprising first and second optimization process, wherein a first optimization process identifies an optimized parameter set for cell growth and a second optimization process identifies an optimized parameter set for a molecular product.

45. The method of claim 44, wherein the optimized parameter set for cell growth informs initial culture conditions for optimizing for the molecular product.

46. The method of claim 1, wherein initial parameter sets comprise randomized, repeated or selected parameter sets.

47. A system comprising:
a) a vessel configured for continuous culture of cells (e.g., a turbidostat or a chemostat);
b) one or more sensors that (i) measure one or more culture outputs of cells being cultured in the vessel, and (ii) transmit the measurements to computer memory;
c) one or more feeds that feed compounds (e.g., nutrients) into the vessel;
d) a computer comprising: (i) a processor; and (ii) a memory, coupled to the processor, and comprising a module comprising:
(1) a dataset comprising, for each of a plurality of culture conditions: measures of culture parameters and measures of culture outputs received from the sensors and associated with the culture conditions;
(2) an automated agent comprising an algorithm that selects a new culture condition predicted to produce an improved measure in the culture output and based on the dataset; and
(3) computer executable instructions that implement the new culture condition in the system, measure the culture output, and add the culture condition and the culture output to the dataset.

48. The system of claim 47, wherein the automated agent uses an artificial intelligence method.

49. The system of claim 48, wherein the artificial intelligence method is selected from reinforcement learning, deep learning, Q-learning, and neural net.

50. The system of claim 47, wherein the automated agent uses a directed search method (e.g., the Nelder-Mead method, a dynamic programming method, a downhill simplex method, a simplex algorithm, linear regression, binary search tree, or random walk).

51. The system of claim 47, comprising one or more of:
A) mixer (e.g., an impeller or a pneumatic agitator) to mix liquid in the vessel, and actuated by a motor;
B) a temperature regulator and a temperature sensor;
C) a source of acidic and alkaline reagents communicating with the vessel interior and a pH meter;
D) an aeration system communicating with the vessel interior and a dissolved oxygen meter;
E) a source of nutrients communicating with the vessel interior and an analyzer for measuring the nutrient
F) an effluent communicating with the vessel interior and a regulatable valve or pump for regulating fluid flow from the vessel;
G) baffles in the vessel;
H) a sparger and mass flow controller communicating with the vessel interior for input of one or more gases and mixtures thereof; and
I) a user interface for communicating instructions with the computer.

52. The system of claim 51, wherein operation of the mixer, the temperature regulator, the source of acidic and alkaline reagents, the source of oxygen and the source of nutrients are under control of the computer.

53. A method comprising:
a) in continuous culture, determining genetic stability of cells in culture;
b) optimizing culture conditions, wherein the culture conditions comprise a parameter set of culture parameters, for a culture output by:
(i) optimizing the culture conditions for cell growth; and/or
(ii) optimizing the culture conditions for the product output;
wherein optimizing comprises performing a set of operations comprising:
I) providing initial measures of cell growth or product output under a plurality of different parameter sets;
II) iteratively performing:
A) based on measures of cell growth or product output, selecting a new parameter set predicted to improve cell growth or product output;
B) perturbing the cells in culture with the new parameter set; and
C) measuring cell growth or product output after perturbing the cells; and
III) selecting an optimized parameter set; and
c) validating the optimized parameter set by:
i) growing the cells in continuous or fed-batch mode under the optimized parameter set; and
ii) measuring the culture output.

54. The method of claim 53, wherein selecting is performed with an automated agent.

55. The method of claim 53, wherein genetic stability is measured as a function of a number of generations until a measure of cell growth or product output significantly deteriorates.

56. The method of claim 53, wherein optimizing for cell growth is performed in a turbidostat and optimizing for culture output is performed in a chemostat.

57. The method of claim 53, wherein optimizing in continuous culture is performed no longer than a period of genetic stability.

58. The method of claim 53, wherein validating is performed at a volume of about 5 liters to about 500 liters.

59. A method comprising:
a) providing a continuous culture of cells in a bioreactor, wherein the culture of cells produces at least one culture output; and
b) while maintaining the cells in continuous culture, using a machine learning optimization method to iteratively alter and test different culture conditions along one or a plurality of different culture parameters for the production of the at least one culture output, wherein a plurality of the iterations alter culture conditions in a direction predicted by the model to improve production of the at least one culture output, wherein the iterations are continued to produce a plurality of improvements.

60. The method of claim 59, wherein the iterations continue until a local maximum of production is reached.

61. The method of claim 59, wherein the iterations are performed between 2 and 25 times, e.g., between 5 and twenty times.

62. The method of claim 59, wherein the iterations are performed at least 5, 6, 7, 8, 9, 10, 11, 12, 13, 14, 15, 16, 17, 18, 19, 20, 21, 22, 23, 24 or 25 times.

63. The method of claim 59, wherein the iterations are performed to produce at least any of 5, 6, 7, 8, 9, 10, 11, 12, 13, 14, 15, 16, 17, 18, 19, 20, 21, 22, 23, 24 or 25 improvements.

64. The method of claim 59, wherein the machine learning optimization method uses reinforcement learning, gradient descent, a genetic algorithm or random search.

65. The method of claim 59, wherein a plurality of iterations comprise altering culture conditions along a plurality of culture parameters.

66. The method of claim 65, wherein the plurality of culture parameters include one or more of pH, temperature, dissolved oxygen, carbon feed rate, and nitrogen feed rate.

67. The method of claim 59, further comprising culturing cells under the improved or optimized culture conditions to produce the culture product, and collecting the culture product from the culture.

68. The method of claim 67, wherein the culture product is produced at a scale of at least any of 500 liters, 1000 liters, 5000 liters, 10,000 liters, 50,000 liters, 100,000 liters, 500,000 liters or 1,000,000 liters.
